(19) United States
(12) Patent Application Publication
(10) Pub. No.: US 2023/0211206 A1
(43) Pub. Date: Jul. 6, 2023

(54) WEARABLE ELECTRONIC DEVICE FOR STAMINA DETERMINATION AND PREDICTION

(71) Applicant: Garmin International, Inc., Olathe, KS (US)

(72) Inventors: Kerri A. Futrell, Mission, KS (US); Julia E. Webster, Overland Park, KS (US); Russell F. Schmidt, Olathe, KS (US); Jordan A. Miller, Olathe, KS (US); Joshua L. Hubbard, Overland Park, KS (US); Mikko J. Seppänen, Jyväskylä (FI); Marika H. Nurmela, Jyväskylä (FI); Maunu M. Toiviainen, Jyväskylä (FI); Titta M. Ollitervo, Jyväskylä (FI); Wilma-Lotta M. Sillanmäki, Helsinki (FI); Sami T. K. Saalasti, Jyväskylä (FI)

(21) Appl. No.: 18/069,809

(22) Filed: Dec. 21, 2022

Related U.S. Application Data
(60) Provisional application No. 63/297,041, filed on Jan. 6, 2022.

(51) Int. Cl. A63B 24/00; A63B 71/06

(52) U.S. Cl. CPC A63B 24/0075; A63B 71/0622; A63B 2024/0065; A63B 2024/0068; A63B 2071/0663; A63B 2230/06

(57) ABSTRACT

An electronic device for measuring parameters related to activities of a user and determining stamina of the user are disclosed herein. A stamina potential metric may be determined from long-term activities that reduce and rebuild stamina potential. A stamina left metric may be determined. The stamina left metric is indicative of short-term work that the user can perform during a workout. The electronic device may track the stamina potential and the stamina left of the user as well as the various parameters associated with the activities performed by the user and track and update the user’s performance metrics. Furthermore, the device may compare a determined amount of stamina required to perform activities with the stamina potential of the user and recommend activities.

### RELATED APPLICATIONS

This non-provisional patent application claims priority benefit, with regard to all common subject matter, of earlier-filed U.S. Provisional Pat. Application No. 63/297,041, filed Jan. 6, 2022, and entitled “DETERMINATION AND DISPLAY OF USER STAMINA.” The identified earlier-filed provisional patent application is hereby incorporated by reference in its entirety into the present application.

### BACKGROUND

Conventional electronic devices, like smartwatches, GPS navigation devices, fitness trackers, etc. utilize sensors and integrate with external devices to measure user activity and provide feedback on distances, times, calorie burn, heart rate, and the like. Such conventional devices have limitations in the user-specific data that can be analyzed.

### SUMMARY

Embodiments of the present disclosure provide a first embodiment directed to a device for determining a stamina reserve of a user. The device comprises at least one storage device storing data indicative of the user and computer-executable instructions, one or more sensors for obtaining activity data associated with the user, a display, and at least one processor configured to execute the computer-executable instructions to perform a method. The method, in some embodiments, comprises accessing, from the at least one storage device, an activity history of the user, determining a stamina potential from at least the activity history of the user, obtaining the activity data from the one or more sensors while the user performs an activity, determining a stamina left metric from the activity data, and causing for display the stamina left metric.

A second embodiment is directed to a wearable device for determining a stamina reserve of a user. The wearable device comprises at least one storage device storing data indicative of the user and computer-executable instructions, one or more sensors for obtaining activity data associated with the user, a display, and at least one processor configured to execute the computer-executable instructions to perform a method. The method comprises accessing, from the at least one storage device, an activity history of the user, determining a stamina potential from at least the activity history of the user, obtaining the activity data from the one or more sensors while the user performs an activity, determining a stamina left metric from the activity data, and causing for display the stamina left metric, wherein the stamina left metric is based on a heart rate measurement and a pulse oximetry data measurement taken while the user is performing the activity.

A third embodiment is directed to wearable device for determining a stamina of a user. The wearable device comprises at least one storage device storing data indicative of the user and computer-executable instructions, one or more sensors for obtaining activity data associated with the user, a display, and at least one processor configured to execute the computer-executable instructions to perform a method. The method comprises accessing, from the at least one storage device, an activity history of the user, determining a stamina potential from at least the activity history of the user, obtaining the activity data from the one or more sensors while the user performs an activity, determining a stamina left metric from the activity data, and causing for display the stamina left metric, wherein the stamina left metric is based on a heart rate measurement and a pulse oximetry data measurement taken while the user is performing the activity and is indicative of an amount of work that the user may perform before short-term energy of the user is exhausted.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the invention will be apparent from the following detailed description of the embodiments and the accompanying drawing figures.

### BRIEF DESCRIPTION OF THE DRAWING FIGURES

Embodiments of the invention are described in detail below with reference to the attached drawing figures, wherein:

FIG. 1 depicts a perspective view of an exemplary mobile electronic device;

FIG. 2A depicts a bottom view of the mobile electronic device;

FIG. 2B depicts electronic components of mobile electronic device for embodiments of the disclosure;

FIGS. 3A and 3B depict components of the mobile electronic device;

FIGS. 4A and 4B depict a display of electronic mobile device for providing an interface to the user;

FIGS. 5A-5F depict exemplary graphics that may be presented to the user for interaction by the display of the mobile electronic device; and

FIG. 6 depicts an exemplary flow chart illustrating a method for embodiments of the disclosure.

The drawing figures do not limit the invention to the specific embodiments disclosed and described herein. The drawings are not necessarily to scale, emphasis instead being placed upon clearly illustrating the principles of the invention.

### DETAILED DESCRIPTION

The following detailed description references the accompanying drawings that illustrate specific embodiments in which the invention can be practiced. The embodiments are intended to describe aspects of the invention in sufficient detail to enable those skilled in the art to practice the invention. Other embodiments can be utilized, and changes can be made without departing from the scope of the invention. The following detailed description is, therefore, not to be taken in a limiting sense. The scope of the invention is defined only by the appended claims, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the technology can include a variety of combinations and/or integrations of the embodiments described herein.

Generally, embodiments of the disclosure are directed to systems and methods for determining a potential stamina and a current stamina of a user. The systems and methods described herein may provide a device for measuring data associated with activity of a user and storing the data for analysis. The data may be obtained from sensors on a device as well as other peripheral sensors. In some embodiments, the peripheral sensors may be associated with machines that the user may be operating. The data may be stored and tracked over time to determine user activity and performance and estimate user ability and predict future performance based on determined metrics.

Embodiments of the present invention relate to an electronic device configured to determine and present information related to a user’s stamina to better inform the user of performance during a physical activity, such as a walk, a run, a bicycle ride or other exercise, general activity, or specific workout. The electronic device utilizes cardiac information, such as heart rate and/or pulse oximetry data (such as SpO2 and a VO2 estimate), as well as any other sensor data to determine the user’s stamina.

To better guide the user with managing the intensity of user’s movements over long physical activities, a stamina potential metric may be determined from the sensor measurements and presented to a user. In embodiments, the stamina potential metric may be presented in a fashion similar to fuel remaining in a vehicle’s fuel tank to enable the user to assess how much stamina the user has left to finish the physical activity. By keeping an eye on the remaining stamina potential, the user can avoid overworking or becoming exhausted before completion of a long physical activity. Various metrics may be presented to the user as described in embodiments below.

In embodiments, the electronic device may determine and provide a stamina left metric associated with an absolute percentage of work the user may continue to perform above a threshold level for the user. The user can use the stamina left metric to determine how much longer the user can perform the physical activity at the current intensity level and gauge the user’s effort during intense physical activities in real time while performing the activity. The stamina left metric can recharge if the user rests or performs the physical activity below the threshold level for the user and may recharge the stamina left metric to the capacity of stamina potential if the user remains below the threshold value long enough. If the stamina potential metric can be thought of as the amount of total fuel in the tank of a vehicle, then the stamina left metric can be thought of as an amount of “turbo boost” remaining for the vehicle.

In some embodiments, the electronic device will determine and present a cruising range time and a cruising range distance, which describe in their respective units a current range of the user. The cruising range time and the cruising range distance may be based on the user continuing at a current intensity or effort level plus remaining energy levels, such as stamina-left..

In some embodiments, the electronic device may determine and present an attack capacity to describe the user’s current ability for a high-intensity effort. The user having a high attack capacity may indicate the user is capable of high-power output, as would be used when “attacking” in a race.

The user’s current stamina (stamina-left) may be represented as a percentage (e.g., 0-100%) and may reflect how much performance capacity the user has left in the tank at the user’s current intensity level or level of effort. The current stamina may combine general fatigue accumulation with more temporary limitations associated with high-intensity efforts like sprints, climbs, and attacks. The user’s potential stamina may also be represented as a percentage (e.g., 0-100%) and may enable the user to monitor the broader, longer lasting effects of muscle cell damage, central nervous fatigue, and carbohydrate (glycogen) depletion. Activities that result in near or total depletion of the user’s potential stamina may typically require several days of recovery before the user may fully recover from the physical activity, which means the user’s potential stamina may not be at 100% at the start of a new activity if the user hasn’t yet fully recovered from the previous activity. The potential stamina may be refilled based on rest, nutrition, sleep, sleep quality, and the like.

In some embodiments, when stamina left is determined by the electronic device as being drained, a bar color can be set color (e.g., red). When the user’s stamina is determined to be recharging, the bar color can be changed to a different color (e.g., green). For any data page and data fields associated with the stamina left and the stamina potential, a recent VO2 estimate and/or SpO2 estimate may be used. The data may be stored in a memory of the electronic device, which may be an electronic wearable device as described in embodiments herein. Furthermore, the data may be determined for the user by the user performing specific activities with a heart rate sensor or any other sensor as described in embodiments herein. In some embodiments, the measurements may be enhanced by providing both heart rate and power measurements from a heart rate sensor and a power sensor paired. In running and walking also GPS, altitude data, wind and running dynamics information – such as vertical oscillation or contact time – may be used to define a grade adjusted pace, VO2-estimate or external power output value.

The metrics may be measured and/or may be calculated based on a history of stored data associated with the user. The history of stored data may be data provided by the user such as, for example, sex, age, height, weight, medical history, nutrition history, VO2max, race records, power records, acclimation status to heat, acclimation status to altitude, and the like. Additionally, the history of stored data may be data obtained from sensors such as, distance, altitude, power , power curve data, heat acclimation, time, heart rate, pulse oximetry information (such as SpO2 and a VO2 estimate), training load, muscle cell damage, central nervous fatigue, and carbohydrate (glycogen) depletion, and the like. In some embodiments, the metrics may be calculated, measured, and/or obtained from the user.

In some embodiments, stamina potential and stamina left may be calculated. Stamina may be quantified as an amount of work performed by the user. As such, stamina may be tracked and quantified based on specific activities that the user performs. Therefore, stamina left may be (or may be indicative of) an amount of work performed by a user during a specific activity. The amount of work performed by the user for the specific activity may be stored for reference and analysis and combined with other activities to determine stamina potential. Both stamina potential and the stamina left metric may be calculated utilizing historical data, such as past activity data for the user. Additionally or alternatively, both stamina potential and the stamina-left metric may be calculated in real-time during activity (e.g., exercise) by the user.

In some embodiments, activities may be determined based on data from various sensors and the determined activities and data from the various sensors may be used to determine stamina potential and stamina left. For example, the user may perform activities such as, for example, running, cycling and various forms of road and offroad biking, spinning, weightlifting, HIIT, yoga, swimming, skiing, skating, surfing, rowing, kayaking, golfing, and playing sports such as basketball, golf, pickleball, tennis, bowling, or the like. In some embodiments, the activities may be general day-to-day activities such as, walking, walking up or down stairs or hills, typing, cooking, driving, sleeping, and the like. The activities may be determined based on stored data indicative of movements of the user and other users.

In some embodiments, stamina left may be stamina potential reduced by a specific amount. For example, the stamina potential may be a total stamina of the user at approximately 100% as determined from previous performances and based on the user’s historic data including, user metrics, nutrition, rest, sleep, quality sleep, and the like. The stamina potential may change based on activities such as eating, walking, exercising and the like. Stamina left may be stamina potential altered by a current short-term exercise that varies by short-term bursts and rests as compared to a threshold value.

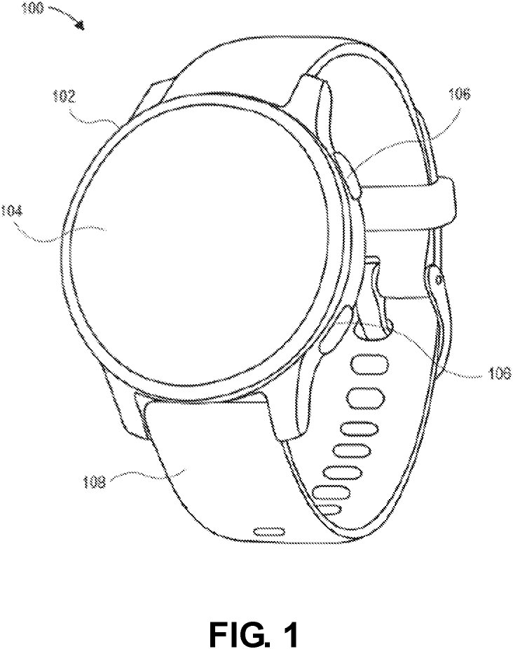

FIG. 1 depicts a perspective view of device 100 in accordance with one or more embodiments of the present disclosure. Device 100 may be a mobile electronic device that may be worn by a user of the device such as, for example, a watch or a wristband. Device 100 may be configured in a variety of ways to determine and provide cardiac information, such as heart rate and pulse oximetry information, as well as user-performance information and navigation functionality to the user of device 100. Device 100 may include housing 102 or a case configured to substantially enclose various components of device 100. Housing 102 may be formed from a lightweight and impact-resistant material such as plastic, nylon, or combinations thereof, for example. Housing 102 may be formed from a conductive material, a non-conductive material, and combinations thereof. Housing 102 may include one or more gaskets, e.g., a seal, to make it substantially waterproof and/or water resistant. Housing 102 may include a location for a battery and/or another power source for powering one or more components of device 100. Housing 102 may be a singular piece or may include multiple sections.

In some embodiments, device 100 includes display device 104 with a user interface. Display device 104 may include a liquid crystal display (LCD), a thin film transistor (TFT), a light-emitting diode (LED), a light-emitting polymer (LEP), and/or a polymer light-emitting diode (PLED). Display device 104 may be capable of presenting text, graphical, and/or pictorial information. Display device 104 may be backlit such that it may be viewed in the dark or other low-light environments. One example embodiment of display device 104 is a 100-pixel by 64-pixel film compensated super-twisted nematic display (FSTN) including a bright white light-emitting diode (LED) backlight. Display device 104 may include a transparent lens that covers and/or protects components of device 100. Display device 104 may be provided with a touch screen to receive input (e.g., data, commands, etc.) from a user. For example, a user may operate device 100 by touching the touch screen and/or by performing gestures on the screen. In some embodiments, the touch screen may be a capacitive touch screen, a resistive touch screen, an infrared touch screen, combinations thereof, and the like. Device 100 may further include one or more input/output (I/O) devices (e.g., a keypad, buttons, a wireless input device, a thumbwheel input device, etc.). The I/O devices may include one or more audio I/O devices, such as a microphone, speakers, and the like. Additionally, user input may be provided from movement of housing 102, for example, an inertial sensor(s), e.g., accelerometer, may be used to identify vertical, horizontal, angular movement and/or tapping of housing 102 or the lens.

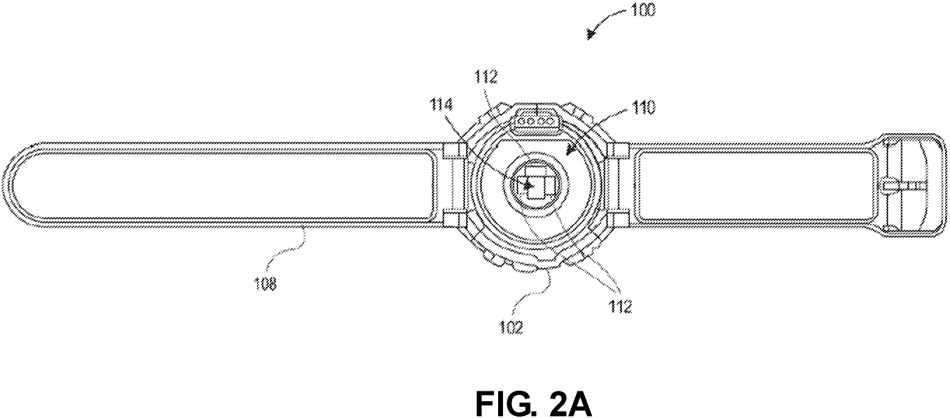

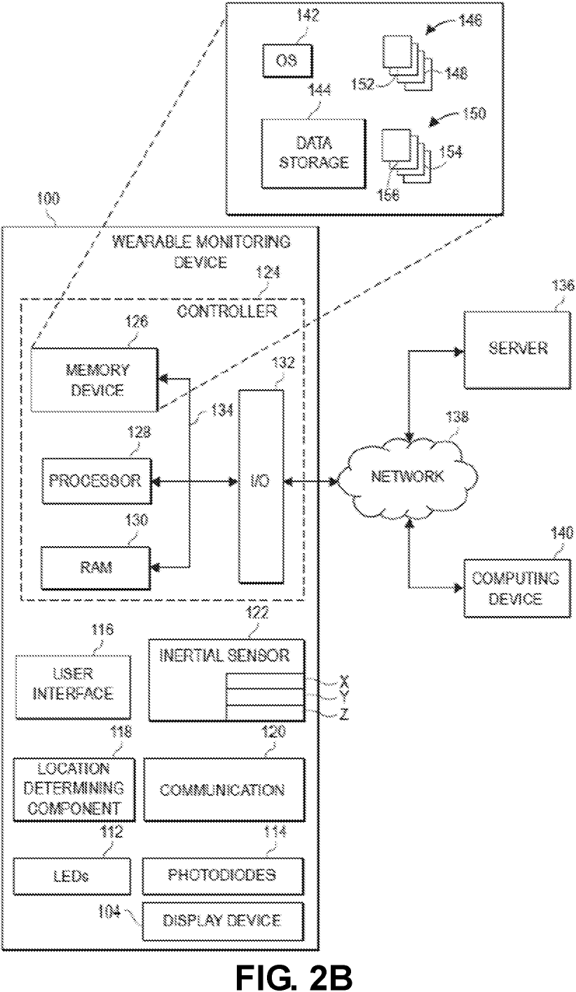

In accordance with one or more embodiments of the present disclosure, the user interface may include one or more control buttons 106. As illustrated in FIG. 1, four control buttons 106 are associated with, e.g., adjacent, the housing 102. While FIG. 1 illustrates four control buttons 106 associated with housing 102, it is understood that device 100 may include a greater or lesser number of control buttons 106. In one embodiment, each control button 106 is configured to generally control a function of device 100. Functions of device 100 may be associated with a location determining component and/or a performance monitoring component as further described below in connection with FIG. 2B. Functions of device 100 may include, but are not limited to, displaying a current geographic location of device 100, mapping a location on display device 104, locating a desired location and displaying the desired location on display device 104, and presenting information based on a physiological characteristic (e.g., heart-rate (HR), heart-rate variability, blood pressure, or SpO2 percentage, for example) or a physiological response (e.g., stress level, body energy level, etc.) of the individual.

FIG. 2A depicts a bottom view of an embodiment of device 100, which may be a wearable device for determining and providing cardiac information, such as heart rate (HR) and pulse oximetry information. Device 100 may also include an optical signal assembly, including one or more emitters (e.g., LEDs 112) of visible and/or non-visible light and one or more receivers (e.g., photodiodes 114) of visible and/or non-visible light that generate a light intensity signal based on the received reflection of light.

Device 100 may include a means for attaching, e.g., a strap 108, that enables device 100 to be worn by the user. When device 100 is worn by the user, one or more LEDs and one or more photodiodes may be securely placed against the skin of the user. Strap 108 may be coupled to and/or integrated with housing 102 and may be removably secured to housing 102 via attachment of securing elements to corresponding connecting elements. Some examples of securing elements and/or connecting elements include, but are not limited to, hooks, latches, clamps, snaps, and the like. Strap 108 may be made of a lightweight and resilient thermoplastic elastomer and/or a fabric, for example, such that strap 108 may encircle a portion of the user without discomfort while securing device 100 to the user. Strap 108 may be configured to attach to various portions of the user, such as the user’s leg, waist, wrist, forearm, upper arm, and/or torso.

FIG. 2B depicts a system diagram showing the components of device 100 for carrying out embodiments of the disclosure. Device 100 includes user interface module 116, a location determining component 118 (e.g., a global positioning system (GPS) receiver, assisted GPS, etc.), communication module 120, inertial sensor 122 (e.g., accelerometer, gyroscope, etc.), and controller 124. Device 100 may be a general-use wearable and mobile computing device (e.g., a watch, activity band, etc.), a cellular phone, a smartphone, a tablet computer, or a mobile personal computer, capable of monitoring a physiological characteristic and/or response of an individual as described herein. Device 100 may be a thin-client device or terminal that sends processing functions to server device 136 via network 138. Communication via network 138 may include any combination of wired and wireless technology. For example, network 138 may include a USB cable between device 100 and computing device 140 (e.g., smartphone, tablet, laptop, etc.) to facilitate the bi-directional transfer of data between device 100 and computing device 140.

Controller 124 may include memory device 126, microprocessor (MP) 128, random-access memory (RAM) 130, and input/output (I/O) circuitry 132, all of which may be communicatively interconnected via address/data bus 134. Although I/O circuitry 132 is depicted in FIG. 2B as a single block, I/O circuitry 132 may include a number of different types of I/O circuits. Memory device 126 may include operating system 142, data storage device 144, a plurality of applications 146, and/or a plurality of software routines 150. Operating system 142 of memory device 126 may include any of a plurality of mobile platforms, such as the iOS®, Android™, Palm® webOS, Windows® Mobile/Phone, BlackBerry® OS, or Symbian® OS mobile technology platforms, developed by Apple Inc., Google Inc., Palm Inc. (now Hewlett-Packard Company), Microsoft Corporation, Research in Motion (RIM), and Nokia, respectively. Data storage device 144 of memory device 126 may include application data for the plurality of applications 146, routine data for the plurality of software routines 150, and other data necessary to interact with the server device 136 through the network 138. In particular, data storage device 144 may include cardiac component data associated with one or more individuals. The cardiac component data may include one or more compilations of recorded physiological characteristics of the user, including, but not limited to, a hemoglobin saturation values, a heart rate (HR), a heart-rate variability (HRV), a blood pressure, motion data, a determined distance traveled, a speed of movement, calculated calories burned, power, body temperature, and the like. In some embodiments, controller 124 may also include or otherwise be operatively coupled for communication with other data storage mechanisms (e.g., one or more hard disk drives, optical storage drives, solid state storage devices, etc.) that may reside within device 100 and/or operatively coupled to network 138 and/or server device 136.

Device 100 also includes an optical signal assembly including one or more light sources, such as LEDs 112. The optical signal assembly also includes one or more light detectors such as photodiodes 114. In some embodiments, LEDs 112 output visible and/or non-visible light and one or more photodiodes 114 receive transmissions or reflections of the visible and/or non-visible light and convert the received light into electrical current, which, in some embodiments, is converted into a digital value by an analog to digital converter. Each LED 112 generates light based on an intensity determined by the processor. For example, LEDs 112 may include any combination of green light-emitting diodes (LEDs), red LEDs, and/or infrared or near-infrared LEDs that may be configured by the processor to emit light into the user’s skin. In some embodiments, the red LEDs operate at a wavelength between approximately 610 nm and 700 nm. In some embodiments, a first LED produces light at approximately 630 nm, a second LED operates at approximately 940 nm, and a third LED operates at approximately 660 nm. The device 100 also includes display device 104 as described in connection with FIG. 1 above.

Device 100 also includes one or more photodiodes 114 capable of receiving transmissions or reflections of visible-light and/or infrared (IR) light output by LEDs 112 into the user’s skin and generating a SpO2 signal based on the intensity of the reflected light received by each photodiode 114. The light intensity signals generated by the one or more photodiodes 114 may be communicated to processor 128. In embodiments, processor 128 includes an integrated photometric front end for signal processing and digitization. In other embodiments, processor 128 is coupled with a photometric front end. The photometric front end may include filters for the light intensity signals and analog-to-digital converters to digitize the light intensity signals into SpO2 signals including a cardiac signal component associated with the user’s heartbeat. In some embodiments, processor 128 may comprise a plurality of processors for performing the various functions described herein.

Typically, when device 100 is worn against the user’s body (e.g., wrist, fingertip, ear, etc.), one or more LEDs 112 are positioned against the user’s skin to emit light into the user’s skin and one or more photodiodes 114 are positioned near LEDs 112 to receive light emitted by the one or more emitters after transmission through or reflection from the user’s skin. Processor 128 of device 100 may receive a SpO2 signal based on a light intensity signal output by one or more photodiodes 114 based on an intensity of light after transmission of the light through or reflection from the user’s skin that has been received by one or more photodiodes 114.

In both the transmitted and reflected uses, the intensity of measured light may be modulated by the cardiac cycle due to variation in tissue blood perfusion during the cardiac cycle. In activity environments, the intensity of measured light may also be strongly influenced by many other factors, including, but not limited to, static and/or variable ambient light intensity, body motion at measurement location, static and/or variable sensor pressure on the skin, motion of the sensor relative to the body at the measurement location, breathing, and/or light barriers (e.g., hair, opaque skin layers, sweat, etc.). Relative to these sources, the cardiac cycle component of the SpO2 signal can be very weak, for example, by one or more orders of magnitude.

In some embodiments, location determining component 118 generally determines a current geolocation of the device 100 and may process a first electronic signal, such as radio frequency (RF) electronic signals, from a global navigation satellite system (GNSS) such as the global positioning system (GPS) primarily used in the United States, the GLONASS system primarily used in the Soviet Union, or the Galileo system primarily used in Europe. Location determining component 118 may include satellite navigation receivers, processors, controllers, other computing devices, or combinations thereof, and memory. Location determining component 118 may be in electronic communication with an antenna (not shown) that may wirelessly receive an electronic signal from one or more of the previously mentioned satellite systems and provide the first electronic signal to location determining component 118. Location determining component 118 may process the electronic signal, which includes data and information, from which geographic information such as the current geolocation is determined. The current geolocation may include geographic coordinates, such as the latitude and longitude, of the current geographic location of the device 100. Location determining component 118 may communicate the current geolocation to processor 128. Generally, location determining component 118 is capable of determining continuous position, velocity, time, and direction (heading) information.

In some embodiments, inertial sensor 122 may incorporate one or more accelerometers positioned to determine the acceleration and direction of movement of device 100. The accelerometer may determine magnitudes of acceleration in an X-axis, a Y-axis, and a Z-axis to measure the acceleration and direction of movement of device 100 in each respective direction (or plane). It will be appreciated by those of ordinary skill in the art that a three-dimensional vector describing a movement of device 100 through three-dimensional space can be established by combining the outputs of the X-axis, Y-axis, and Z-axis accelerometers using known methods. Single and multiple axis models of the inertial sensor 122 are capable of detecting magnitude and direction of acceleration as a vector quantity and may be used to sense orientation and/or coordinate acceleration of the user.

The optical signal assembly (including LEDs 112 and photodiodes 114), location determining component 118, and inertial sensor 122 may be referred to collectively as the “sensors” of device 100. It is also to be appreciated that additional location determining components 118 and/or inertial sensor(s) 122 may be operatively coupled to device 100. Device 100 may also include or be coupled to a microphone incorporated with user interface module 116 and used to receive voice inputs from the user while device 100 monitors a physiological characteristic and/or response of the user determines physiological information based on the cardiac signal.

Communication module 120 may enable device 100 to communicate with the computing device 140 and/or server device 136 via any suitable wired or wireless communication protocol independently or using I/O circuitry 132. The wired or wireless network 138 may include a wireless telephony network (e.g., GSM, CDMA, LTE, etc.), one or more standard of the Institute of Electrical and Electronics Engineers (IEEE), such as 802.11 or 802.16 (Wi-Max) standards, Wi-Fi standards promulgated by the Wi-Fi Alliance, Bluetooth standards promulgated by the Bluetooth Special Interest Group, a near field communication standard (e.g., ISO/IEC 18092, standards provided by the NFC Forum, etc.), ANT, and so on. Wired communications are also contemplated such as through universal serial bus (USB), Ethernet, serial connections, and so forth.

Device 100 may be configured to communicate via one or more networks 138 with a cellular provider and an Internet provider to receive mobile phone service and various content, respectively. Content may represent a variety of different content, examples of which include, but are not limited to map data, which may include route information; web pages; services; music; photographs; video; email service; instant messaging; device drivers; real-time and/or historical weather data; instruction updates; and so forth.

User interface module 116 of device 100 may include a “soft” keyboard that is presented on display device 104 of device 100, an external hardware keyboard communicating via a wired or a wireless connection (e.g., a Bluetooth keyboard), and/or an external mouse, or any other suitable user-input device or component. As described earlier, user interface module 116 may also include or communicate with a microphone capable of receiving voice input from a vehicle operator as well as display device 104 having a touch input.

With reference to controller 124, it should be understood that controller 124 may include processor 128, which may be multiple microprocessors, multiple RAMs 130 and multiple memory devices 126. Controller 124 may implement RAM 130 and memory devices 126 as semiconductor memories, magnetically readable memories, and/or optically readable memories, for example. Processor 128 may be one or more processors and may be adapted and configured to execute any of the plurality of applications 146 and/or any of the plurality of software routines 150 residing in the memory device 126, in addition to other software applications. One of the plurality of applications 146 may be client application 152 that may be implemented as a series of machine-readable instructions for performing the various functions associated with implementing the performance monitoring system as well as receiving information at, displaying information on, and transmitting information from device 100. Client application 152 may function to implement a system wherein the front-end components communicate and cooperate with back-end components as described above. Client application 152 may include machine-readable instructions for implementing a graphical user interface by user interface module 116 to allow a user to input commands to, and receive information from, device 100. One of the plurality of applications 146 may be native web browser 148, such as Apple’s Safari®, Google Android™ mobile web browser, Microsoft Internet Explorer® for Mobile, Opera Mobile™, that may be implemented as a series of machine-readable instructions for receiving, interpreting, and displaying web page information from the server device 136 or other back-end components while also receiving inputs from the device 100. Another application of the plurality of applications 146 may include native web browser 148 that may be implemented as a series of machine-readable instructions for receiving, interpreting, and displaying web page information from server device 136 or other back-end components within client application 152.

A plurality of applications 146 (here, client applications) or software routines 150 may include an accelerometer routine 154 that determines the acceleration and direction of movements of the device 100, which correlate to the acceleration, direction, and movement of the user. Accelerometer routine 154 may receive and process data from inertial sensor 122 to determine one or more vectors describing the motion of the user for use with client application 152. In some embodiments where inertial sensor 122 includes an accelerometer having X-axis, Y-axis, and Z-axis accelerometers, accelerometer routine 154 may combine the data from each accelerometer to establish the vectors describing the motion of the user through three-dimensional space. In some embodiments, accelerometer routine 154 may use data pertaining to less than three axes.

Plurality of applications 146 or software routines 150 may further include velocity routine 156 that coordinates with location determining component 118 to determine or obtain velocity and direction information for use with one or more of the plurality of applications, such as client application 152, or for use with other routines.

The user may also launch or initiate any other suitable user interface application (e.g., native web browser 148, or any other one of the plurality of applications 146) to access server device 136 to implement the monitoring process. Additionally, the user may launch client application 152 from device 100 to access server device 136 to implement the monitoring process.

After the above-described data has been gathered or determined by the sensors of device 100 and stored in memory device 126, device 100 may transmit information associated with measured cardiac information, such as heart rate (HR), pulse oximetry (SpO2) information, blood oxygen saturation percentage (pulse oximetry signal), peak-to-peak interval (PPI), heart-rate variability (HRV), motion data (acceleration information), location information, stress intensity level, and body energy level of the user to computing device 140 and server device 136 for storage and additional processing. For example, in embodiments where device 100 is a thin-client device, computing device 140 or server device 136 may perform one or more processing functions remotely that may otherwise be performed by device 100. In such embodiments, computing device 140 or server device 136 may include a number of software applications capable of receiving user information gathered by the sensors to be used in determining a physiological response (e.g., a stress level, an energy level, etc.) of the user. For example, device 100 may gather information from its sensors as described herein, but instead of using the information locally, device 100 may send the information to computing device 140 or server device 136 for remote processing. Computing device 140 or server device 136 may perform the analysis of the gathered user information to determine a stress level or a body energy level of the user as described herein. Server device 136 may also transmit information associated with the physiological response, such as a stress level, an energy level, of the user. For example, the information may be sent to computing device 140 or server device 136 and include a request for analysis, where the information determined by computing device 140 or server device 136 is returned to device 100.

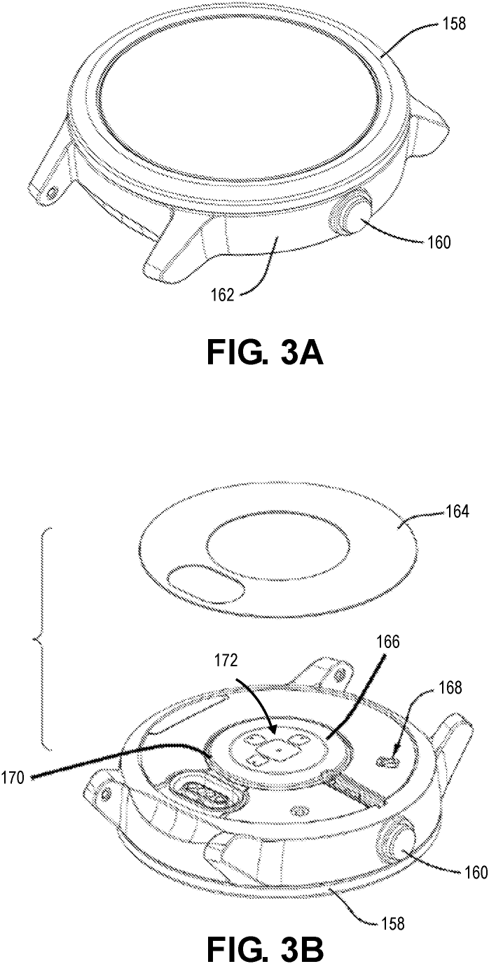

In some embodiments, device 100 (as further depicted in FIGS. 3A and 3B) may generate and graphically display an electrocardiogram (ECG) waveform of an electrical activity of a wearer’s heart. The electronic device may utilize two or more contact points 172 to receive electrical bio signals (electrocardiogram signals) from the wearer, from which the electrocardiogram waveform is generated as depicted in FIG. 3B. For instance, a first contact point may be located on an underside of the electronic device where it may be in generally constant contact with the skin of the wearer’s wrist and a second contact point may be bezel 158 or a portion of bezel 158 that may also function as an antenna, or push-button 160, which may be control button 106, that may also depress or rotate to provide a user input interface enabling access of additional functionality. Alternatively, two contact points may be located on an underside of the electronic device where it may be in generally constant contact with the skin of the wearer’s wrist and a third contact point may be bezel 158 or a portion of bezel 158 that may also function as an antenna, or push-button 160 that may also depress or rotate to provide a user input interface enabling access of additional functionality.

Broadly characterized, embodiments of device 100 may include three contact points, a processing element, and a display. Two of the three contact points may be located on an underside (a bottom surface) of device 100 where each contact point may be in constant contact with the skin of a wearer’s wrist when worn by a user. In embodiments, the third contact point may be an electrically conductive bezel 158 (or a portion of the bezel 158) that functions as one or more antennas for device 100. For instance, bezel 158 may provide at least a portion of an antenna coupled with a location determining element. Bezel 158 or portion thereof may be accessible to receive a touch from a user’s finger or thumb (of the opposite hand) to initiate the sensing and monitoring of the electrical activity of the wearer’s heart. In particular, device 100 may be configured to perform the location determining function and the heart monitoring function simultaneously or it may be configured to switch between these functions, in which case the function of bezel 158 may be selected by processor 128 to correspond to a desired function. Additionally, or alternatively, the third contact point may be pushbutton 160 that is accessible to receive a touch from a user’s finger or thumb (of the opposite hand) to initiate the sensing and monitoring of the electrical activity of the wearer’s heart. Pushbutton 160 may also be depressed or rotated to access and/or initiate additional general, fitness, or non-fitness-related functionality of device 100.

Processor 128 may be a general or dedicated processing element configured to receive a first electrical bio signal (electrocardiogram signal) from the first contact point, a second electrical bio signal (electrocardiogram signal) from the second contact point and a third electrical bio signal (electrocardiogram signal) from the third contact point. The processing element may be configured to determine the electrical activity of the wearer’s heart based on electrical bio signals received through the three contact points once physical contact is made between the wearer’s wrist and the contact points. Processor 128 may generate, and store in memory device 126, electrocardiogram data based on the electrocardiogram waveform and generate an electrocardiogram image based on the stored electrocardiogram data. Processor 128 may be further configured to control display device 104 to present determined electrical activity as an electrocardiogram waveform image, a sequence of single waveform images, or a stream of multiple waveform images.

For example, outer back plate 164 may form a portion of lower wall 168 of housing 102 and one of the three contact points. Outer back plate 164 may be formed from an electrically conductive material, may have substantially any suitable shape, and may be located on a lower surface of lower wall 168 of housing 102, so that it is generally in constant contact with the wearer’s skin. Inner back plate 166 may form a portion of lower wall 168 of housing 102 and a second of the three contact points. Inner back plate 166 may also be formed from an electrically conductive material, may have substantially any suitable shape, and may also be located on a lower surface of lower wall 168 of the housing 102, so that it is generally in constant contact with the wearer’s skin. In embodiments, annular ring 170 formed of a non-conductive material and annular ring 170 may electrically isolate outer back plate 164 from the inner back plate 166. Outer back plate 164 and inner back plate 166 may each be configured to receive an electrical bio signal (electrocardiogram signal) via the wearer’s skin, and to provide the electrical bio signal to processor 128.

In embodiments, lower wall 168 of housing 102 may not be continuous, but may include an opening of circular, square, rectangular, or other geometric shape. An upper wall of housing 102, which may be formed by the bezel 158, generally opposes the lower wall and may include an upper surface. In some embodiments, the upper surface may further include an opening of circular, square, rectangular, or other geometric shape. The internal cavity of housing 102 may contain and/or retain many of the other components of device 100. The lower wall of housing 102 may have a round, circular, or oval shape with a single circumferential side wall, while in other embodiments, the lower wall may have a four-sided shape, such as a square or rectangle, or other polygonal shape, with housing 102 including four or more sidewalls 162. The upper wall may generally match the shape of the lower wall 168 of housing 102.

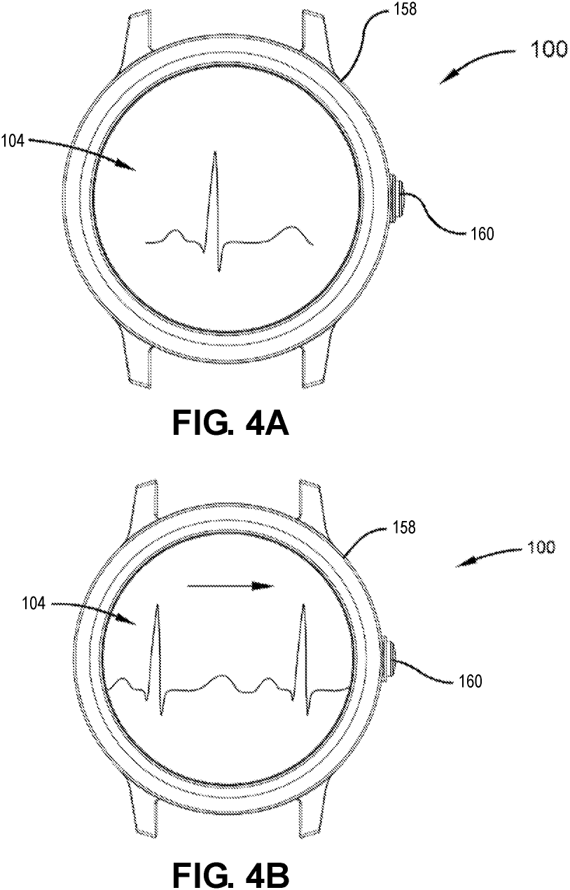

FIG. 4A depicts a plan view of device 100 displaying a sequence of single electrocardiogram images and FIG. 4B is a plan view of device 100 displaying a stream of multiple electrocardiogram images. Processor 128 may control display device 104 to present the electrocardiogram waveform as one or more electrocardiogram images. In some embodiments, as shown in FIG. 4A, processor 128 may control display device 104 to present a sequence of electrocardiogram images, where each electrocardiogram image corresponds to one sequence of heartbeats of the wearer for a period of time. In other embodiments, processor 128 may control display device 104 to present a stream of electrocardiogram images, wherein the stream of electrocardiogram image corresponds to (a plurality of) heartbeats of the wearer and the electrocardiogram images are scrolled such that a current or most-recently generated electrocardiogram image is continuously presented on display device 104. The direction of streaming may be indicated by an arrow, as shown in FIG. 4B.

The disclosed techniques and described embodiments may be implemented in a wearable monitoring device having a housing implemented as a watch, a mobile phone, a hand-held portable computer, a tablet computer, a personal digital assistant, a multimedia device, a media player, a game device, or any combination thereof. The wearable monitoring device may include a processor configured for performing other activities.

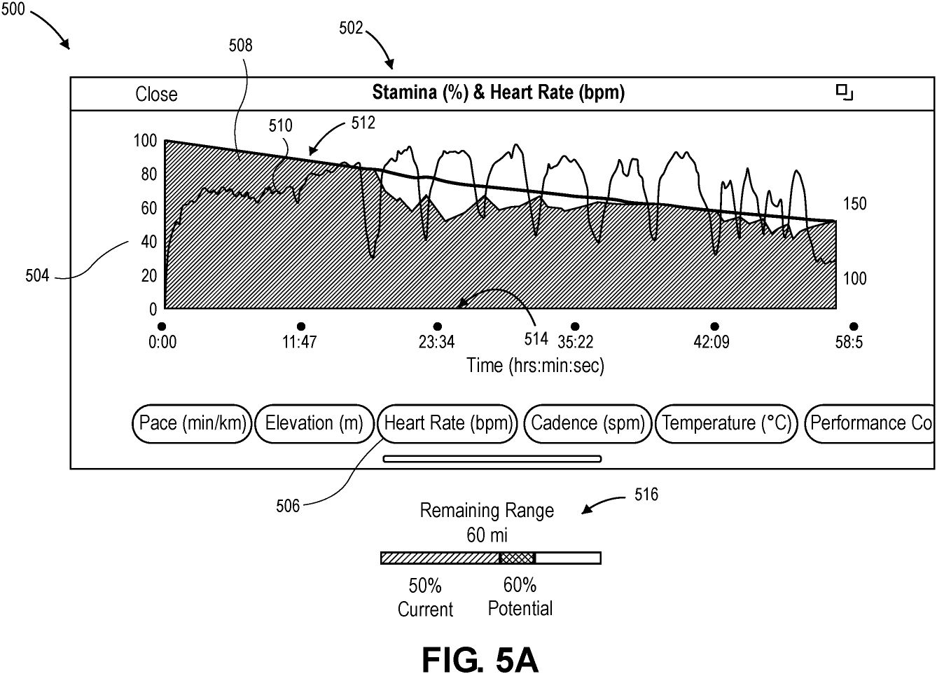

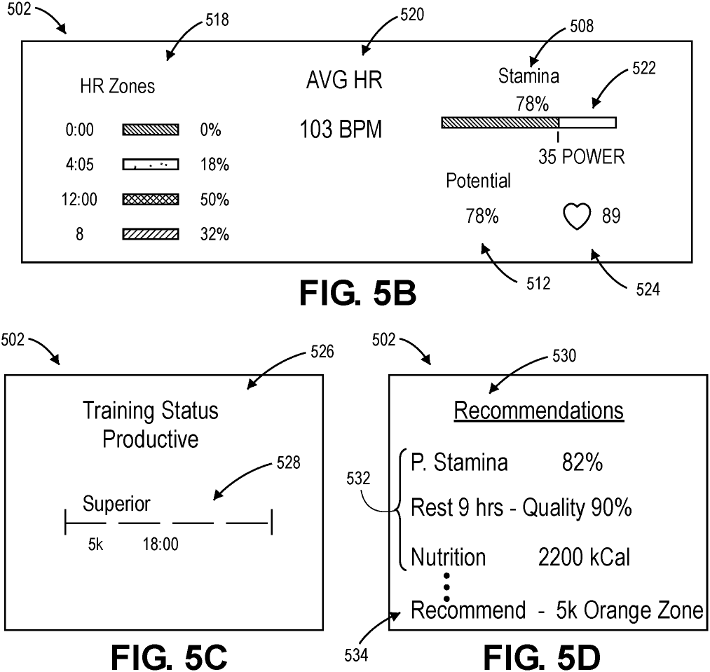

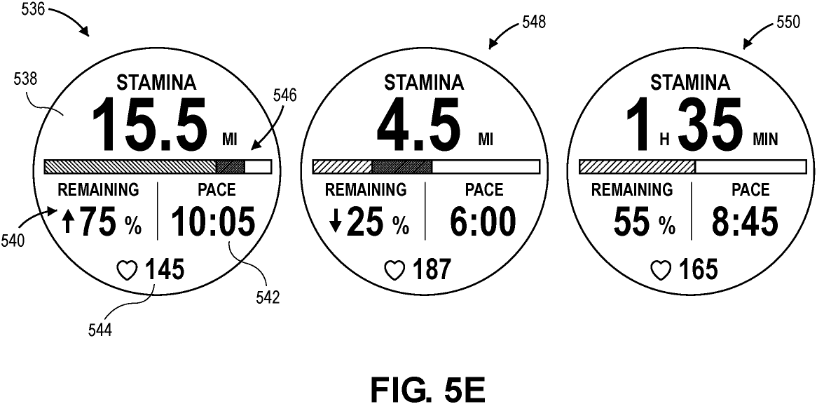

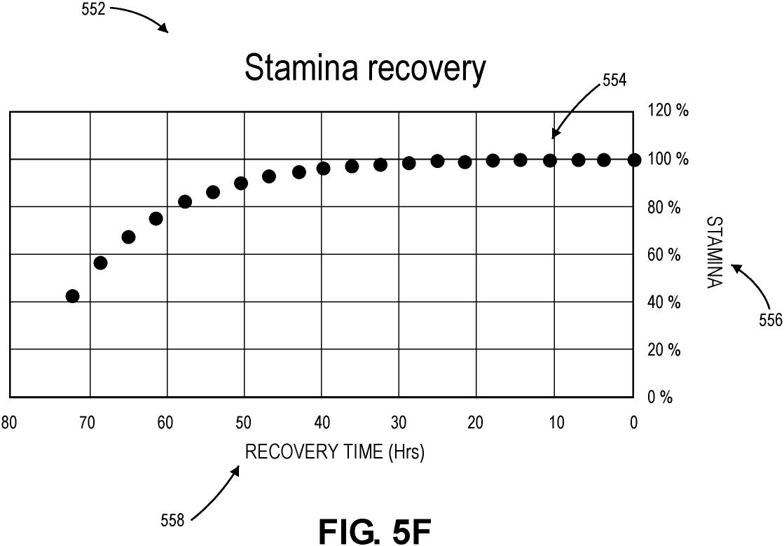

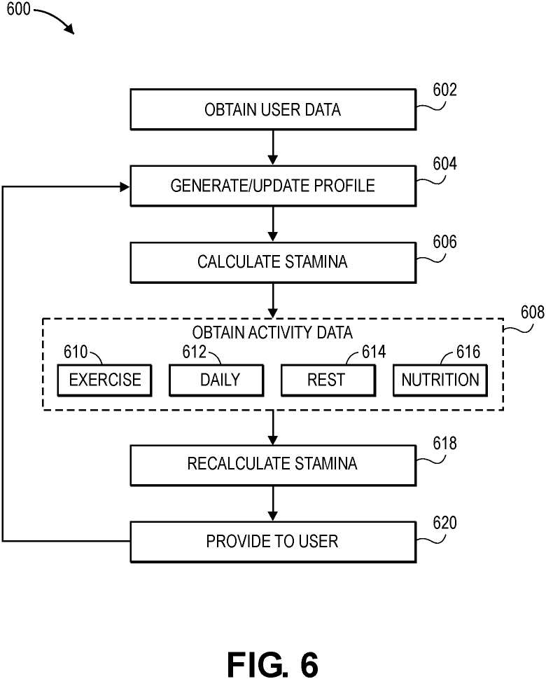

FIGS. 5A-5D depict an interface 500 that may be presented on display device 104 of device 100 for interaction with the user and displaying information related to the user’s activities and performance. The above-described systems of device 100 shown in FIGS. 1-4B may be utilized to perform the functions and display the graphics as described below in reference to FIGS. 5A-6. The interactions with the user may include displaying information and receiving input through the various buttons and switches of the exemplary electronic device described above or through any input by the touchscreen of the device 100.

In some embodiments, analytics associated with the user’s activities during a time range may be displayed. Any activities including workouts, steps, sedentary time, rest, nutrition, or the like may be displayed to the user in any table, graph, or easy to understand graphic. Exemplary workout analytics are shown in FIG. 5A. Graphic 502 includes a table comprising dependent variable 504 that may be any set of values including a standardized set of values (e.g., percentage) such that a plurality of variables may be represented by graphic 502. Furthermore, independent variable 514 may be displayed. Independent variable 514 here is time; however, independent variable 514 may be any variable described herein. As depicted, graphic 502 displays potential stamina metric 512, current stamina metric (Stamina left) 508 and heart rate metric 510. Similarly, or alternatively, any of various metrics 506 (including any metric described herein) may be determined and provided by graphic 502. Exemplary metrics may be stamina potential and stamina left, power, location, speed, pace, elevation, heart rate, cadence, air temperature, user body temperature, and any other metric. The metrics displayed on graphic 502 may be customizable by the user.

The exemplary metrics may be obtained from any of the above-described sensors associated with device 100 and/or obtained from peripheral devices associated with machines used in workouts communicatively connected sensors or device 100 such as smartphones, tablets, computers, and the like. For example, a metric that may be used as a component in determining stamina may be user power output. Power output may be determined by peripheral sensors associated with workout machines such as, for example, treadmills, bicycles, stationary bikes, ellipticals, and the like. If the user is running instead of riding, pace and altitude may be used to inform stamina instead of power level.

The data presented by graphic 502 may be representative of a workout as depicted in FIG. 5A. Here, stamina potential metric 512, current stamina metric 508, and heart rate metric 510 may be displayed to the user via device 100. As shown, the heart rate is tracked during the workout on the same graphic 502 such that the user may easily see and track their progress throughout the workout. The user may switch between various metrics displayed on graphic 502 by interacting with interface 500 on display device 104.

The user may initiate the starting time, or the system may detect that a workout has begun by obtaining information from various sensors. In some embodiments, the user may input a specific activity or workout that the user plans to perform. Device 100 may track the workout adjusting stamina potential metric 512 and stamina left metric 508 accordingly. In some embodiments, stamina potential may be a long-term calculation that is tracked over time. The user may go day after day performing various tasks such as sleeping, eating, working, exercising, and the like. The activities may be tracked and combined to determine potential stamina. As described above, potential stamina may be an amount of power or indicative of an amount of work that may be performed by the user over time. For example, stamina potential may be a long-term ongoing metric that may be adjusted over time. In some embodiments, stamina potential may be a long-term workout or may represent an amount of energy/power/work (and corresponding units) that a user has to perform activities before rest and nutrition are required for the user’s body to continue performing activities. The stamina potential may be calculated based on a combination of the user’s activities over time such as, one day, two days, one week, one month, one year, or the like. Any time range may be tracked, and the stamina potential may be reduced and increased according to the activities performed by the user. In other examples, stamina potential may be calculated in real-time based on current activity data of the user, including for instance information such as heart rate provided by one or more sensors.

In some embodiments, stamina potential may be reduced based on recent exercise activities. For example, the user may perform a workout such as, for example, cycling. As shown in FIG. 5A stamina potential metric 512 decreases as the workout continues over time. Stamina potential metric 512 may be indicative of a total amount of energy that the user has. The potential stamina may be long-term energy that may take longer recovery activities to increase such as sleep, nutrition, and the like.

Stamina left may increase and decrease across a shorter timeline. Stamina left may be representative of an amount of work that the user may be able to perform based on real-time activities and metrics such as heart rate and pulse oximetry data (such as SpO2 and a VO2 estimate). The heart rate and pulse oximetry data may be compared to historical data associated with the user. For example, in one hypothetical example, the user may typically sustain a heart rate of 100 bpm during their workout. If the user goes above that to, for example, 120 bpm, the user’s stamina left metric 508 may drop causing the user to not be able to finish their workout at the current intensity. The user may then decrease their intensity of the workout resulting in a reduced heart rate of 90 bpm for a time range. However, in other examples, lactate threshold values and ranges may be employed instead of, or in addition to, heart rate to provide a better representation of user effort. Furthermore, as the user’s stamina left metric 508 is determined across a plurality of workouts and time ranges, the threshold value for HR may change according to the user’s changing performances.

As shown in FIG. 5A the heart rate metric 510 showing the measured heart rate of the user may significantly move up and down based on the workout of the user. The user may perform high intensity work such as sprints causing the heart rate to jump to HR zones of aerobic (70-80%) or anaerobic (80-100%), then take breaks between each sprint allowing the heart rate to return to near normal HR zones. Stamina left metric 508 may be quickly affected by these changes while stamina potential metric 512 decreases more linearly. This may be the case, for example, because stamina left metric 508 represents the relatively short-term response of the body to activities such as sprinting and recovery and is more highly influenced by short term events such as high heart rate and rest intervals, while stamina potential metric 512 represents data over a longer time range. Therefore, stamina potential metric 512 may appear as a more linear decline while performing a workout.

In some embodiments, the electronic device will determine and present a cruising range time and a cruising range distance, which describe in their respective units a current range or endurance of the user. The cruising range time and the cruising range distance may be based on the user continuing at a current intensity or effort level plus remaining energy levels, such as stamina potential or stamina left. Cruising range distance 516 is depicted along with the instantaneous stamina left metric 508 of 50% and the instantaneous stamina potential metric 512 of 60%. The stamina and cruise range distance/time may be provided to the user as a colored bar and using numbers showing distance and stamina percentages providing the user with an easily digestible visual while training.

FIG. 5B depicts exemplary graphic 502 that may be displayed to the user showing a workout that the user is performing or has just finished. Graphic 502 may display HR zones 518 displaying various colored bars representing HR zones 518 based on age or any other user-specific attribute. HR zones 518 may be indicative of intensity of a workout based on heart rate such as, for example low intensity, temperate, aerobic, anaerobic, very light, light, moderate, hard, maximum, and the like. In some embodiments, HR zones 518 below the heart rate threshold value, as described above, may represent recovery. The HR zones 518 below the heart rate threshold rates are where the user recovers or the user’s stamina left metric 508 increases. The user maintaining a heart rate above the threshold may result in stamina left metric 508 being diminished based on higher-intensity work. The HR zones 518 may be displayed in real time showing the user’s HR zones 518 for the workout in which they are performing. Furthermore, the percentage of time spent in each HR zone 518 may also be displayed.

In some embodiments, average heart rate 520 for a workout may be shown. Average heart rate 520 may be shown along with stamina left metric 508 showing the user their performance metrics during and after the workout. Stamina left metric 508 may be displayed on the bar graphic. Here, power metric 522 is also displayed. Power may be determined from a power sensor associated with a machine, such as a bicycle. In some embodiments, power output of the user may also be determined from pace.

Furthermore, stamina potential metric 512 is shown as 78% and stamina left metric 508 is also shown as 78% while current heart rate 524 is 89. These metrics may indicate that the workout is complete, and the user has recovered as stamina left metric 508 and stamina potential metric 512 are equal and current heart rate 524 is near resting heart rate.

FIG. 5C depicts exemplary workout status 526 that provides feedback to the user for the workout. For example, the workout here was a 5k and status tracker 528 shows that the status of the workout is superior based on the 18:00 minute 5k. As such, the user is performing at a superior level relative to other performances in the history of the user, other users, general population data, combinations thereof, and/or the like.

In some embodiments, device 100 may provide recommendations 534 in the form of recommendations chart 530 to the user based on the stamina potential of the user. FIG. 5D depicts an exemplary graphic 502 displaying exemplary contributing metrics 532 to stamina potential metric 512 for providing recommendations 534. For example, preloaded workouts or courses via maps data (e.g., Open Maps, Google Maps) may be stored by device 100. Device 100 may access the course or workout data and determine an estimated amount of stamina that may be required to finish the workout or the course. The historic data may comprise data from other users and/or data associated with the workout such as hills, stairs, workout activities (e.g., pushups, sit ups, squats), and the like. Furthermore, the historic data may include past performances by the user on the workout or course. As such, the stamina potential metric 512 of the user may be directly compared to an estimated amount of stamina required to complete the workout or the course.

Device 100 may take into account past activities such as, for example, recent workouts, daily activities such as walking, working, and the like, rest, rest quality, and nutrition (e.g., calorie intake, carbohydrate intake, sugar intake, salt intake, water intake, supplement intake, and the like). As shown in FIG. 5D, based on a rest quality of 90%, an intake of 2,200 Calories and other factors, stamina potential is 82% and the recommended workout for the user is 5k while averaging a heart rate in the Orange HR zone. Furthermore, in some embodiments, the user may select a workout or a series of workouts and device 100 may evaluate the workouts and determine if the user can do them or if other workouts should be recommended.

In some embodiments, the user may input, and/or the device 100 or associated service may create a week of workouts and device 100 can calculate a maximum effectiveness or optimization of the workouts to increase performance based on the workout history of the user and the increase in performance. For example, a user may perform a workout on a first day leaving a 50% stamina potential while the next workout requires 70% stamina potential. As such, the user may have to wait a day to recover before performing the next workout. Device 100 may calculate the heart rate or intensity level necessary to maintain during the first workout such that the next day the user will have 70% stamina potential and will be able to perform the second workout. This provides the user the ability to exercise more and more often rather than having to wait to recover because they overworked during an individual exercise or workout.

Generally, stamina feedback can tell a user when they are overreaching and depleting their energy too quickly such that the user will not be able to complete the workout. Further, stamina feedback can tell the user if they are not pushing enough. Device 100 may determine and display stamina left metric 508. Furthermore, device 100 may determine and display a range of stamina left values at specific times such that the user may stay on track in their workout . This may assist a user in pacing themselves during workouts and across several workouts to get the most from their exercises.

Furthermore, by showing the stamina left metric 508 device 100 may assist the user in realizing when they begin to fatigue and recognize the feelings of fatigue. Therefore, the user may better understand their body and their fitness and performance levels.

In some embodiments, the user may define user objectives based on performance, weight loss, or the like. As such, based on the user history and course and workout optimization described above, an optimized schedule may be created for the user to reach the desired objective. For example, the user may wish to decrease their pace from 11 minutes per mile to 10 minutes per mile. Based on the history of performance increase of the user, device 100 may determine a running and exercise schedule for the user to reach their goal in the shortest amount of time. This may work by maximizing training time by optimizing potential stamina for each workout each day. As described above, it may be the case that a user may overwork themselves and need rest for several days to recover, thereby missing workouts that could have been done if the training plan had been optimized. For example, the user may run 1 hour every other day for 7 days. The same user may be able to run 40 minutes every day without a day of rest in between. Therefore, after seven days, the user running every other day runs 4 hours and the user running everyday runs 4.6 hours. This simplified calculation shows that a user’s stamina may be optimized for each workout each day such that the user may exercise more often to achieve their goals in minimal time.

FIG. 5E depicts an example stamina display 536 comprising activity indicator 538, current stamina indicator 540 (e.g., remaining stamina) as a percent of the potential, pace indicator 542, heart rate indicator 544, and stamina bar 546. Stamina display 536 may be displayed by display device 104. The user may interact with display device 104 to customize stamina display 536. Activity indicator 538 may display total distance, remaining distance, total time, remaining time, time active, or any metric indicative of a current workout as shown by distance activity indicator 548 and time activity indicator 550. Activity indicator 538 may be programmed to display a metric indicative of the activity by the user or the user may input an activity and the distance or time may be automatically displayed. In some embodiments, the activity indicator may change based on the sensors described herein. For example, the user may run a marathon and the distance left may be displayed together with cruising range which may allow user to ensure that cruising range doesn’t drop below remaining distance. Similarly, or alternatively, the user may begin a stored workout, or a workout associated with a third-party application stored on the device 100.

Current stamina indicator 540, in some embodiments, present the current stamina to the user. The current stamina indicator 540 may be indicative of a stamina left from the potential stamina during a workout and may be presented as a percentage, a total amount, a bar graph, a line graph, or the like. In some embodiments, stamina bar 546 may also show the current stamina. Stamina bar 546 may be broken into various sections as shown in FIG. 5E. The various sections may represent, potential stamina, current stamina, and stamina required to complete the workout at the current pace.

In some embodiments, pace indicator 542 may provide a current pace or an average throughout the workout. This provides the user feedback to maintain a pace to complete the workout without overexerting themselves. Furthermore, heart rate indicator 544 may be provided to the user. The heart rate indicator may allow the user to see what heart rate zone they are in and modify their pace to meet their needs.

In some embodiments, the user may be provided with an optimized pace or power profile for a specific route. An objective may be, for example, finishing the route without severe fatigue or just simply optimizing the race time so that user will achieve best possible finishing time. This kind of optimization can be done either before an event or during event when the race route and user’s physical abilities are known. Optimization may utilize information on weather conditions on the route - including for example wind speed & direction, temperature, solar radiation intensity and humidity. In addition to the elevation profile - also information on absolute elevation may be utilized. For example -user may get significantly lower power targets for a given route profile if the event is held in high altitude or conditions with high heat and humidity. Wind conditions may also affect so that route with headwind will have longer estimated finishing time which will further lower the average pace or power levels that will be provided to the user. In all such embodiments it may be useful to use information on current stamina or stamina potential at the starting point or any other point of exercise to be able to adjust pace or power targets for the remainder of the route. As described in the previous embodiments -parameters such as previous activity history (both previous activities and the current activity so far) nutrition, rest, stress, sleep, sleep quality , and like, may impact stamina level.

FIG. 5F depicts an exemplary stamina recovery chart 552 that may be displayed by graphic 502 on interface 500 by display device 104. Stamina recovery chart 552 may display stamina recovery curve 554 with stamina as the dependent variable 556 as a percentage, for example, and recovery time as the independent variable 558 in time (e.g., minutes, hours, days, and weeks). Stamina may be recovered based on recovery activities such as rest, rest quality, nutrition, and the like as described above. Recovery time may be determined during activities based on stamina left, stamina potential, activity duration, activity intensity, and the like.

Stamina recovery curve 554 may comprise a curve slope at any point that may be different based on the rest and nutrition of the user. As described in embodiments above, if the nutrition is high, such as maintaining a well-balanced diet according to the Food and Drug Administration (FDA) and/or tracking protein, carbohydrates, sugar, salt, water, vitamins, and minerals, as well as any other item that may affect stamina recovery, the slope of the curve may be higher reaching 100% more quickly. Furthermore, the user may take certain supplements that decrease recovery time. These supplements may be included in nutrition and increase the slope of the curve further reducing recovery time as well as increasing stamina potential. Stamina potential may also be calculated based on rest. If the user rests more or has high quality rest or sleep, the curve slope may be increased resulting in a quicker rise in stamina potential. Furthermore, each person is different and recovers at their own rate. The activities of the user may be tracked over time and the stamina potential recovery may be indicative of the specific user’s ability to recover after workouts and perform better over time.

FIG. 6 depicts a flow chart 600 for an exemplary method of determining stamina of a user. At step 602, device 100 may obtain user data. The user data may be input by the user and may be indicative of characteristics of the user such as health conditions, age, weight, height, and past performance metrics such as fastest mile, sports played, timelines, and the like. Furthermore, the user may input general nutrition information such as how often the user eats certain foods and supplements and the like. In some embodiments, the user may also input goals and activities that the user likes and dislikes.

At step 604, electronic device 100 may generate a user profile for the user based on the obtained user data. The user profile may store all data associated with the user and may be updated regularly and automatically periodically or when it is determined that the user has performed an activity that affects the stamina calculations and the user performance calculations.

At step 606, device 100 may determine stamina potential. The user profile may determine an initial stamina potential based on the user data. Furthermore, the user profile including the stamina potential may be updated by any data obtained by device 100. In some embodiments, customized schedules may be generated and updated to meet user goals by optimizing workouts to maintain estimated stamina potential metrics 512 as described in embodiments above. Furthermore, workout recommendations may be determined based on the determined stamina potential. The stamina potential, customized schedules, and recommendations may be stored in the user profile.

At steps 608, 610, and 612 device 100 may obtain activity data via any one of a plurality of sensors. The sensors may be any heart rate sensor, pulse oximetry meter, accelerometer, GPS sensor, power sensor, and the like. The various exemplary sensors may detect data and the detected data may be used to calculate distance, time, heart rate, pulse oximetry information (such as SpO2 and a VO2 estimate), muscle cell damage, central nervous fatigue, and carbohydrate (glycogen) depletion, and the like. Furthermore, the sensors may detect and calculate performance metrics associated with known activities such as golf, walking, hiking, climbing, running, and the like.

At step 614, device 100 may obtain rest activity. The user may lay down to rest or have a specific rest or sleep schedule. Device 100 may detect movements of the user during these times to determine a quality of sleep or rest. The quality of sleep or rest may be used to along with other metrics to determine how quickly the user’s stamina potential increases.

At step 616, device 100 may obtain nutrition activity. The user may input foods eaten during the day and the device 100 or a third-party app associated with device 100 or an auxiliary device may determine nutrition information associated with the foods. The nutrition information such as, for example, calories, carbohydrates, fat, sugars, salt, as well as vitamins and minerals may be added in to determine stamina potential for the user.

At step 618, electronic device 100 may determine changes in stamina potential and stamina left based on the obtained activity data. The stamina calculations may be performed while the activities at step 606 are performed by the user. For example, the user may perform any workout as described above. The data associated with the workout may be obtained and stamina potential metric 512 and stamina left metric 508 may be calculated in real time to provide the user with the data and analytics by display device 104 in step 620 and as described above.

Although the invention has been described with reference to the embodiments illustrated in the attached drawing figures, it is noted that equivalents may be employed, and substitutions made herein without departing from the scope of the invention as recited in the claims.

Having thus described various embodiments of the invention, what is claimed as new and desired to be protected by Letters Patent includes the following:

1. A device for determining a stamina of a user, the wearable device comprising:
at least one storage device storing data indicative of the user and computer-executable instructions;
one or more sensors for obtaining activity data associated with the user;
a display; and
at least one processor configured to execute the computer-executable instructions to:
access, from the at least one storage device, an activity history of the user;
determine a stamina potential from at least the activity history of the user;
obtain the activity data from the one or more sensors while the user performs an activity;
determine a stamina-left metric from the activity data; and
cause display of the stamina-left metric .

2. The device of claim 1, wherein the stamina potential is determined in real time from the activity data and displayed to the user.

3. The device of claim 1, wherein the stamina-left metric is based on a heart rate measurement taken while the user is performing the activity and is indicative of an amount of work that the user may perform before short-term energy of the user is exhausted.

4. The device of claim 1, wherein the at least one processor is further configured to execute instructions to recommend to the user an adjustment to an intensity level of the activity based on the stamina-left metric.

5. The device of claim 1, wherein the at least one processor is further configured to execute instructions to recommend a workout schedule for the user to optimize at least one of the stamina potential and the stamina-left metric to meet a user-defined goal.

6. The device of claim 1, wherein the at least one processor is further configured to execute instructions to:
receive, from the user, a new activity to be performed; and
determine an estimated stamina required to perform the new activity based on the activity history of the user and determining if the user has enough potential stamina to perform the activity.

7. The device of claim 6, wherein the at least one processor is further configured to execute instructions to suggest an alternative activity to be performed based on a comparison of the stamina potential and the estimated stamina required to perform the new activity.

8. The device of claim 1, wherein the wearable device is a watch or a wristband.

9. A device for determining a stamina of a user, the wearable device comprising:
at least one storage device storing data indicative of the user and computer-executable instructions;
one or more sensors for obtaining activity data associated with the user;
a display; and
at least one processor configured to execute the computer-executable instructions to:
access, from the at least one storage device, an activity history of the user;
determine a stamina potential from at least the activity history of the user;
obtain the activity data from the one or more sensors while the user performs an activity;
determine a stamina-left metric from the activity data; and
cause display of the stamina-left metric,
wherein the stamina-left metric is based on a heart rate measurement taken while the user is performing the activity.

10. The device of claim 9, wherein the stamina-left metric is indicative of an amount of work that the user may perform before short-term energy of the user is exhausted.

11. The device of claim 9, wherein the at least one processor is further configured to execute instructions to receive peripheral data from at least one peripheral device associated with a machine associated with the activity of the user.

12. The device of claim 11, wherein the at least one processor is further configured to execute instructions to:
calculate a power output of the user from the peripheral data;
wherein the at least one peripheral device associated with the machine is a power sensor and the machine is a bicycle.

13. The device of claim 9, wherein the at least one processor is further configured to execute instructions to:
determine a cruising range distance indicative of an estimated distance that the user is able to travel during the activity based on a current intensity level of the user; and
determine a cruising range time indicative of an estimated time that the user is able to travel during the activity based on the current intensity level of the user.

14. The device of claim 13, wherein the current intensity level is based on at least a measured heart rate of the user.

15. The device of claim 9, wherein the at least one processor is further configured to execute instructions to recommend an adjustment to an intensity level of the user based on the stamina-left metric.

16. The device of claim 9, wherein the at least one processor is further configured execute instructions to recommend a workout schedule for the user to optimize at least one of the stamina potential and stamina-left metric to meet a user-defined goal.

17. A device for determining a stamina of a user, the wearable device comprising:
at least one storage device storing data indicative of the user and computer-executable instructions;
one or more sensors for obtaining activity data associated with the user;
a display; and
at least one processor configured to execute the computer-executable instructions to:
access, from the at least one storage device, an activity history of the user;
determine a stamina potential from at least the activity history of the user;
obtain the activity data from the one or more sensors while the user performs an activity;
determine a stamina-left metric from the activity data; and
cause display of the stamina-left metric,
wherein the stamina-left metric is based on a heart rate measurement taken while the user is performing the activity and is indicative of an amount of work that the user may perform before short-term energy of the user is exhausted.

18. The device of claim 17, wherein the at least one processor is further configured to execute instructions to:
receive a new activity to be performed and determining an estimated stamina required to perform the activity based on the activity history of the user; and
determine if the user has enough potential stamina to perform the activity.

19. The device of claim 18, wherein the at least one processor is further configured to execute instructions to suggest an alternative activity to be performed based on a comparison of the stamina potential and an alternative estimated stamina required to perform the new activity.

20. The device of claim 17, wherein the at least one processor is further configured to execute instructions to recommend a workout schedule for the user to optimize at least one of the stamina potential and the stamina-left metric to meet a user-defined goal.
